(19) United States
(12) Patent Application Publication
(10) Pub. No.: US 2018/0169434 A1
(43) Pub. Date: Jun. 21, 2018

(54) Treatment of Neurodegenerative Diseases Using Light Therapy

(71) Applicant: Erchonia Corporation, Melbourne, FL (US)

(72) Inventor: Steven C. Shanks, Melbourne, FL (US)

(73) Assignee: Erchonia Corporation, Melbourne, FL (US)

(21) Appl. No.: 15/604,363

(22) Filed: May 24, 2017

Related U.S. Application Data
(60) Provisional application No. 62/435,326, filed on Dec. 16, 2016.

(51) Int. Cl. A61N 5/06

(52) U.S. Cl. CPC A61N 5/0622; A61N 2005/0643; A61N 2005/067; A61N 2005/0651; A61N 2005/0642

(57) ABSTRACT

A non-invasive method for treating neurodegenerative diseases. Light energy is applied externally to a patient's head to stimulate different neurological pathways, reduce inflammation and stimulate mitochondria function in the brain. The light is applied near the area of the brain that is malfunctioning or that is associated with the source of the disease to be treated. The treatment can be enhanced by activating the cranial nerves while the light is applied.

The wavelength of the applied light range from about 400-760 nm, and in a preferred embodiment is about 640 nm. The applied light energy can be applied with a pulse frequencies that mimic healthy brain function of alpha, beta, delta, and theta waves. The pulse frequencies can be applied series, alternately, or simultaneously. The light can be emitted from the same light emitter or from multiple emitters and is preferably laser light.

### CROSS-REFERENCE TO RELATED APPLICATIONS

This application claims the benefit of co-pending U.S. Provisional Application No. 62/435,326 filed Dec. 16, 2016.

### FIELD OF INVENTION

This invention relates generally to methods for treating brain disease. This invention relates more particularly to treating brain disease non-invasively using light therapy.

### BACKGROUND

Neurodegenerative diseases occur when nerve cells in the brain or peripheral nervous system lose function over time and may ultimately die. Neurodegenerative disease is a broad category of brain diseases including autism spectrum disorder; Alzheimer's disease; amyotrophic lateral sclerosis (“ALS”); Creutzfeldt-Jakob disease; vascular dementia; Lewy body dementia; fronto-temporal dementia; multi-infarct dementia; vitamin B-12 deficiency syndrome; hypothyroidism; Huntington's disease; Parkinson's disease; normal pressure hydrocephalus; and tauopathies. Many types of neurodegenerative disease are progressive, in which symptoms gradually worsen over time, and can be fatal. Many of these brain diseases involve inflammation and the body's overall inflammatory response.

Neurodegenerative disease is a common problem in older demographics, causing sufferers to have significant cognitive decline with accompanying increase in cost of care and burden on caregivers. With an ageing population, the problem is likely to worsen. The causes of neurodegenerative diseases are not well known and although there are many studies underway for the treatment of the disease and its symptoms, there is no cure. Current available medications treat the symptoms, but often have unwanted side-effects. It would be desirable to treat patients suffering from neurodegenerative diseases in order to minimize the symptoms, stop the progression of the disease, and ideally to cure it.

Low-level laser therapy (“LLLT”) has been shown through numerous clinical studies and regulatory clearances to be a safe and effective, simple, non-invasive and side-effect free alternative to medication and surgical procedures for the reduction of symptoms in a variety of conditions. LLLT reduces edema, improves wound healing, and relieves pain of various etiologies. It is also used in the treatment and repair of injured muscles and tendons. Application of LLLT has been shown to have the potential to alter cellular metabolism to produce a beneficial clinical effect. Based on its ability to modulate cellular metabolism and alter the transcription factors responsible for gene expression, LLLT has been found to alter gene expression, cellular proliferation, intra-cellular pH balance, mitochondrial membrane potential, generation of transient reactive oxygen species and calcium ion level, proton gradient and consumption of oxygen. LLLT stimulation of the mitochondria via low-energy light has been shown to provoke a dynamic shift in the function of an individual cell. Laser therapy has been shown to stimulate cell regeneration and later gene expression.

It is an object of this invention to apply light energy to a patient's head to treat neurodegenerative disease.

### SUMMARY OF THE INVENTION

Light energy is applied externally to a patient's head to stimulate different neurological pathways, reduce inflammation and stimulate mitochondria function in the brain. Preferably the light is applied to the scalp on the skull near the area of the brain that is malfunctioning or to the part of the brain associated with the source of the disease to be treated. Due to the systemic effects of applying light anywhere to the brain, the application of light on any area of the head will work on any malady to some degree, but application near the damaged area of the brain is optimal. For an inflammation-based disease such as Parkinson's, the more areas of the head that can be treated the more effective treatment is. The treatment can be enhanced by activating the cranial nerves while the light is applied.

The wavelengths of the applied light range from about 400-760 nm. In a preferred embodiment the applied light is about 640 nm. In a preferred embodiment, the applied light energy is applied with a pulse frequency or frequencies that mimic healthy brain function of alpha, beta, delta, and theta waves. The pulse frequencies can be applied series, alternately, or simultaneously. The light can be emitted from the same light emitter or from multiple emitters. Preferably the light emitted is laser light.

### BRIEF DESCRIPTION OF THE DRAWINGS

FIG. 1 illustrates a portable, floor-supported light-emitting device for treating a patient's head.

FIG. 2 illustrates the light-emitting device of FIG. 1 employing a translucent cap around a patient's head.

FIG. 3 illustrates a wall mounted light-emitting device for treating a patient's head.

FIG. 4 is a schematic illustration of a brain inside a patient's skull.

FIG. 5 is a schematic illustration of a brain.

FIG. 6 is a prior-art table of cranial nerves.

### DETAILED DESCRIPTION OF THE INVENTION

This invention is a method for treating brain disease non-invasively by applying light therapy through a patient's scalp and skull to his brain, cranial nerves, or both. There are a number of variables in light therapy, including the wavelength of the light, the power of the light source, the area impinged by the light, the shape of the beam spot when the light impinges the treated area, the pulse frequency, the intensity or fluence of the light energy, and the treatment duration. The setting of these variables typically depends heavily on the disease, brain, skull, and tissue characteristics of the specific patient. The success of each therapy depends on the relationship and combination of these variables. For example, as disclosed in more detail below, Alzheimer's disease may be treated with one regimen utilizing a given power, wavelength, pulse frequency and treatment duration, whereas autism may be treated with a regimen utilizing a different power, wavelength, pulse frequency and treatment duration, and either regimen may be further adjusted for a given patient depending on that patient's size, weight, age, and stage of the disease.

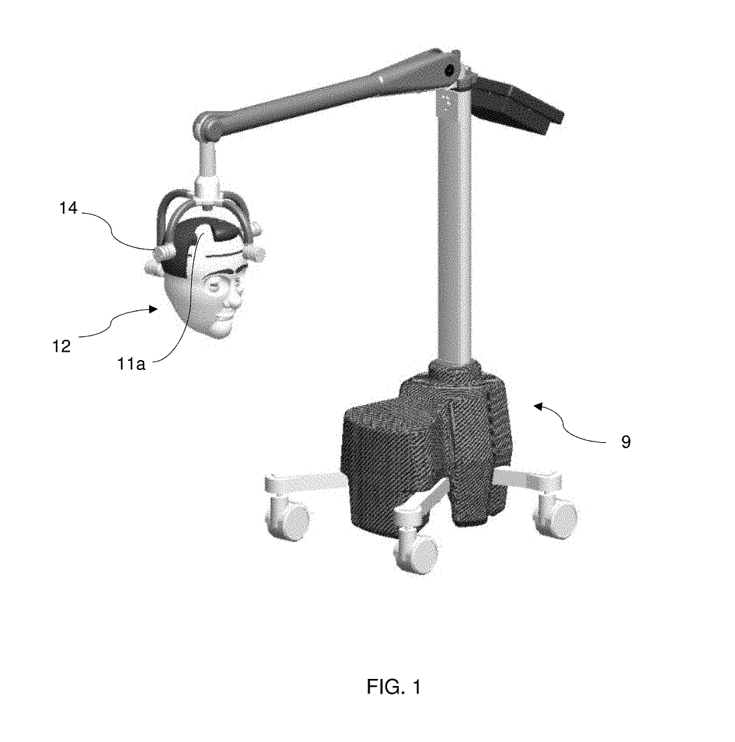

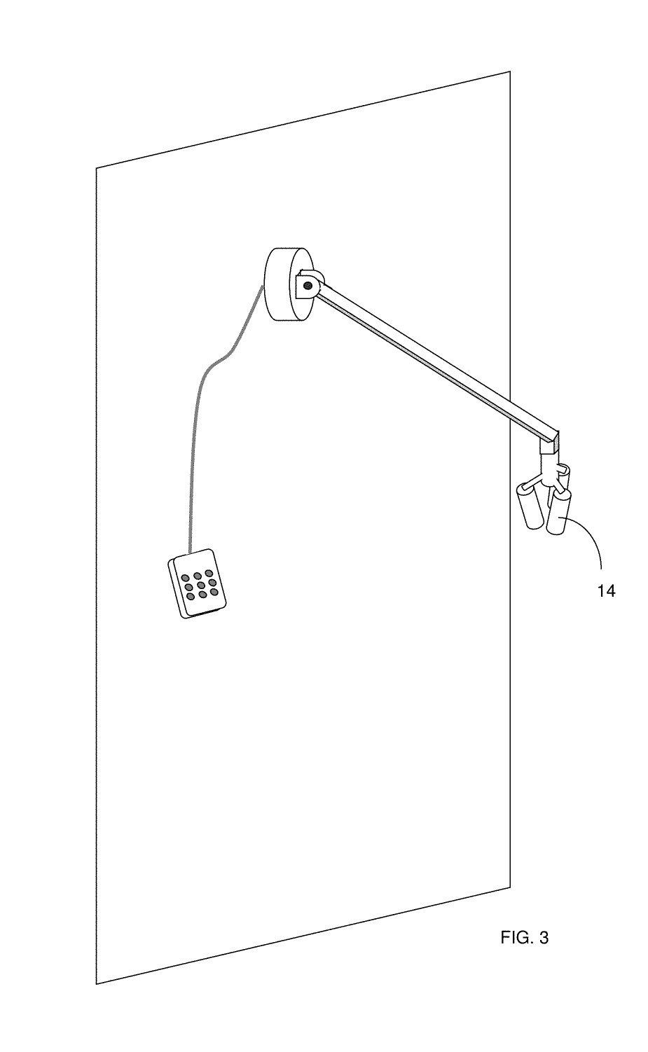

The wavelengths of the light that can be applied range from about 400-760 nm nominal, with the desired wavelength within the spread from nominal. In some embodiments multiple wavelengths are used, either in series, alternately, or simultaneously. The light can be from any source including light-emitting diodes, hard-wired lasers, or laser diodes, but preferably is from a semiconductor laser diode such as Gallium Aluminum Arsenide (GaAlAs) laser diodes, emitting red laser light at 640 nm nominal. Commercial semiconductor laser diodes have a spread of ±10 nm from nominal so the light applied is within the spread from nominal. FIGS. 1, 2, 3 and 5 illustrate probes 14, each containing a laser diode.

The applied light is low-level, typically from emitters of less than 1 W. This low-level light therapy has an energy dose rate that causes no immediate detectable temperature rise of the treated tissue and no macroscopically visible changes in tissue structure. Consequently, the scalp tissue impinged by the light, the skull, and the brain and nerve tissue are not heated and are not damaged.

The applied light energy is applied with a pulse frequency or frequencies of brain waves emanating from a healthy brain, as measured by electroencephalography. Brain waves are neural oscillations in a rhythmic or repetitive neural activity that includes the following:

| Wave | Approximate Frequency | Main Source Location |
|---|---|---|
| Type | Range in Humans | on the Human Brain |
| Delta | 0.5 to 3 Hz 0.5-4 | thalamus or cortex |
| Theta | 3 to 8 Hz | hippocampus |
| Alpha | 8-12.5 Hz | occipital lobe |
| Mu | 7.5-12.5 (and primarily | motor cortex |
| | 9-11) Hz | |
| Beta | 12.5 to 38 Hz | posterior brain |
| Gamma | 38 to 100 Hz | all areas of brain |

Other types of oscillatory activity are found in a healthy central nervous system, and light therapy may be applied at a pulse frequency that mimics that oscillatory activity. Multiple pulse frequencies can be applied series, alternately, or simultaneously. In one embodiment, the light therapy is applied using several light sources, each having a different frequency.

Light is applied to the patient's head 11. The light may be applied to a patient's shaved skull 11a, through the patient's hair, or through a translucent skull cap 20 which may also aid in orienting the light to the desired location on the patient's head. Typically the patient is treated while the patient is vertical or nearly vertical, as opposed to prone or supine, so that all regions of the skull and brain stem can be treated without moving the patient. The patient can be awake, sedated, or asleep.

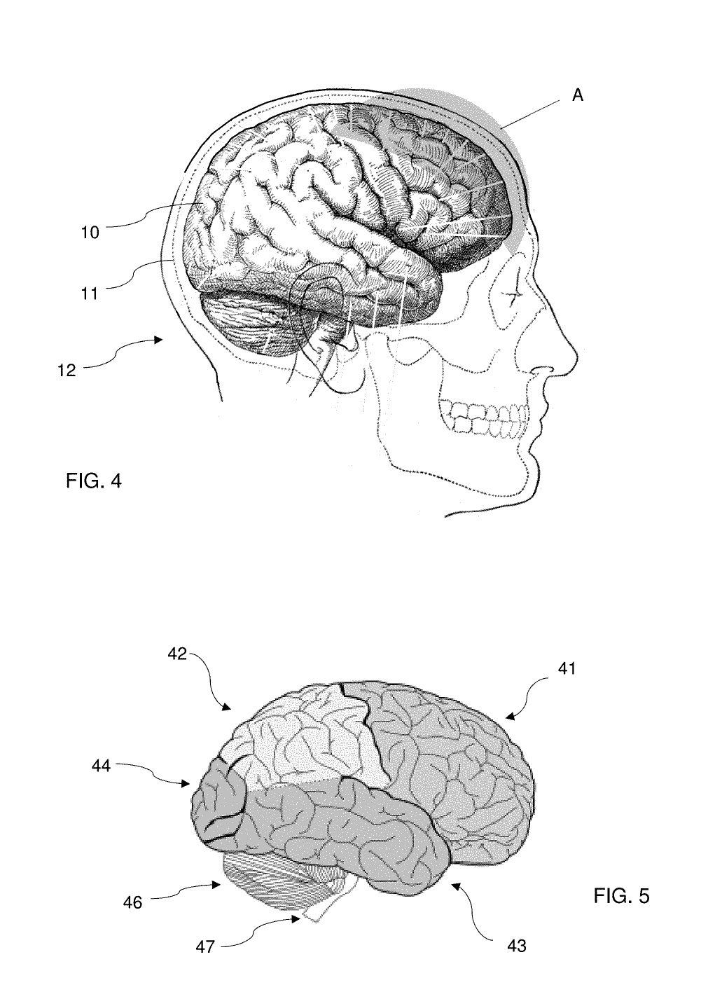

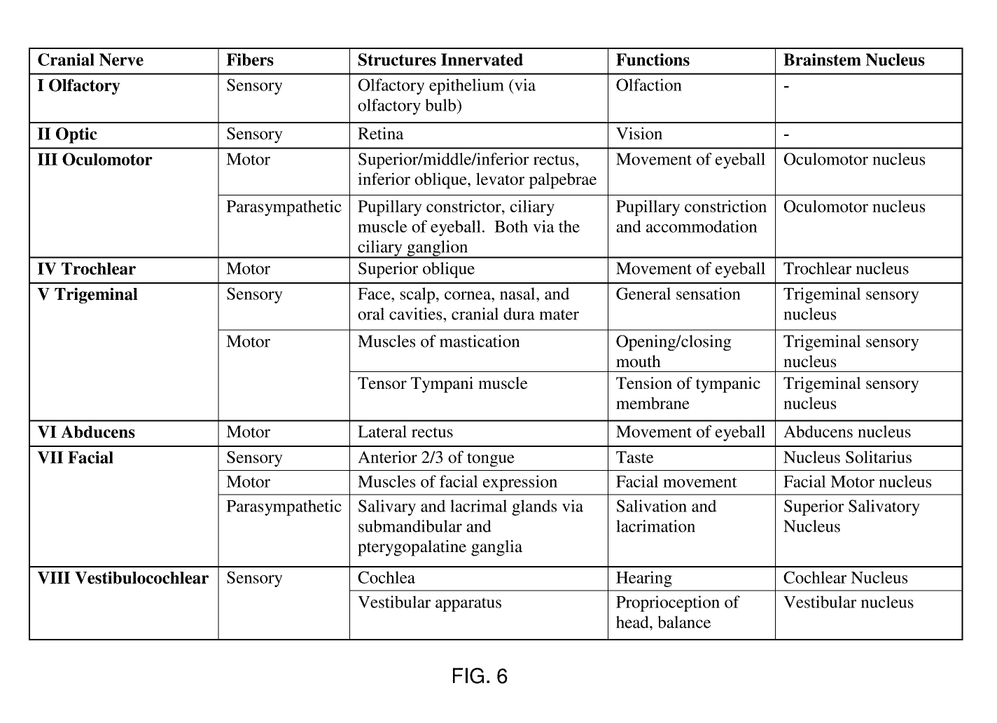

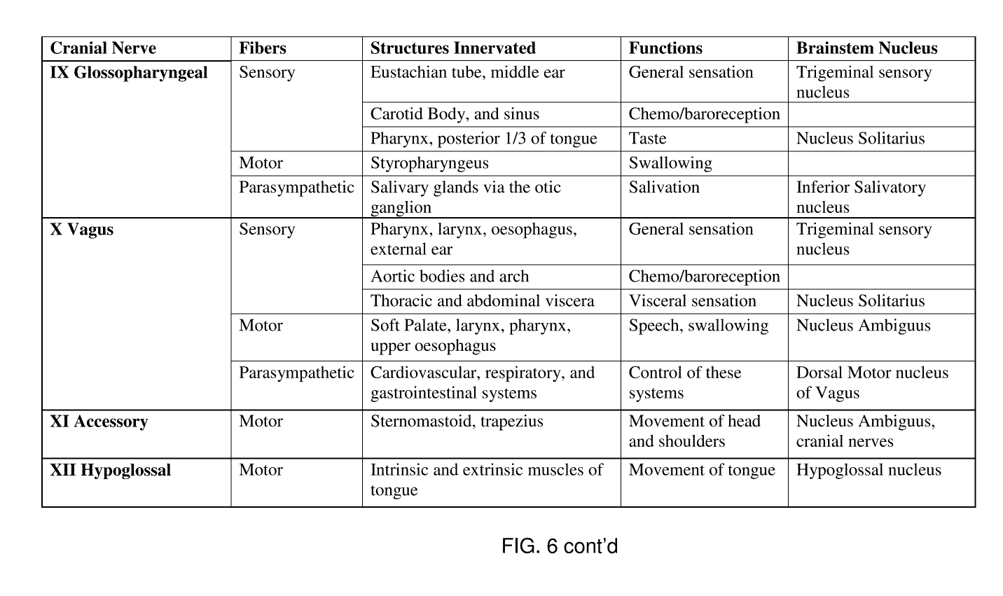

The method is non-invasive. The light is applied to the skull near the area of the brain or cranial nerves (or both) that is malfunctioning or associated with the source of the malady to be treated, if the area is known. As used herein, light applied “near the” area means light applied to the scalp at a position mapped to the area of the brain to be treated, such as the frontal 41, parietal 42, temporal 43, and occipital 44 lobes; the cortex; cerebellum; the brain stem; or where one or more cranial nerves enters the brain. See FIG. 5. For example, if the light is to be applied “near the frontal cortex,” it will be applied to the scalp above the frontal cortex, as indicated generally by area A in FIG. 4. In another example, if the light is to be applied “near the area” of the basal ganglia, which is in the center of the brain, the light will be applied to the scalp all around the head from about the ear lobes up to the top of the head. The treatment can be enhanced by activating the cranial nerves while the light is applied. FIG. 6. Is a table of the cranial nerves and their functions. A cranial nerve is activated by having the patient to execute the function indicated the table of FIG. 6. For example, to activate the olfactory nerve, the patient would be given something with an odor or scent to smell. Similarly, to activate the trochlear nerve, the patient would move his eyeballs.

[symbol] The following table sets forth some of the locations to be treated for specific diseases:

| | Main location to be treated |
|---|---|
| Disease | on the human brain |
| Alzheimer's | frontal cortex, temporal lobe and base |
| Amyotrophic lateral sclerosis | motor cortex |
| (“ALS”) | |
| Autism spectrum disorder | frontal cortex, temporal lobe and |
| | base of skull |
| Epileptic seizures | cortex |
| Huntington's disease | basal ganglia |
| Parkinson's disease | midbrain |
| Pick's disease and other fronto- | frontal lobe |
| temporal dementias | |

Some diseases are not area-specific. That is, some diseases are not necessarily due to a specific damaged area of the brain, instead occurring throughout the brain, or at a different location for each patient, such as Creutzfeldt-Jakob disease, hypothyroidism, Lewy body dementia, normal pressure hydrocephalus, tauopathies, and vascular dementia. For example, with vascular dementia, blood vessels through the brain may be affected, some more than others. For these maladies the light is applied all over the head. For an inflammation-based disease such as Parkinson's, the more areas of the brain that can be treated the more effective treatment is. In fact, due to the systemic effects of applying light anywhere to the brain, the application of light on any area of the skull will work on any malady to some degree, and application to multiple areas is often beneficial. In one embodiment the treatment is applied to a specific hemisphere of the brain. For some diseases the treatment is applied to acupuncture points on the brain.

Another variable in the light treatment parameters is the intensity or fluence of the light energy. U.S. Pat. No. 7,118,588, also incorporated here by reference, discloses a line generator for laser light application. At 4 inches away from the scalp, the 70 degree line generator as disclosed therein creates a projected beam that is 14 cm long and 3 mm wide for a total beam area of 420 mm². The size of each treatment area is the same as the beam profile because the diode is not moved during procedure administration. Given a 600 second treatment time with a 7.5 mW laser, the total energy each produced by each independent laser diode is 4.5 joules per laser. The fluence per laser is calculated as 0.011 J/cm². Using a laser with 5 independent laser diodes, as shown in FIG. 1, each laser diode treats a different (separate) areas of the head, so the fluence remains the same (unchanged) at 0.011 joules; however, the total energy delivered to the subject per 10-minute procedure administration across all 5 laser diodes and respective treatment areas combined is 22.5 joules.

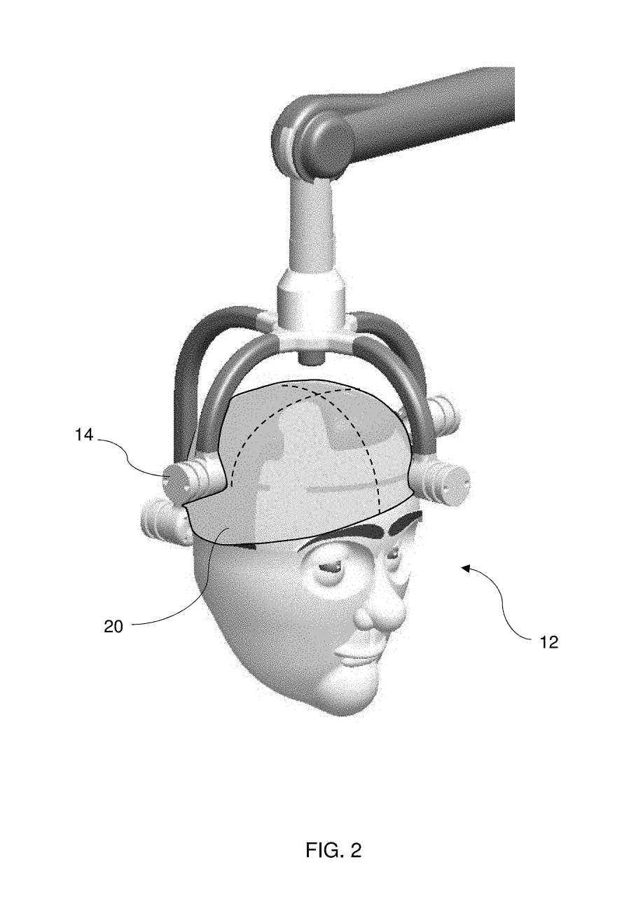

The laser energy can be applied to the patient using a variety of laser devices, such as a hand-held laser device, a full-body laser scanner, a wall-mounted laser device, or a stand-alone laser device. Handheld lasers are described in U.S. Pat. Nos. 6,013,096 and 6,746,473, which are incorporated herein by reference. A full-body laser scanner is described in U.S. Pat. No. 8,439,959, incorporated herein by reference. Wall-mount and stand-alone lasers 9 are described in U.S. Pat. No. 7,947,067 as illustrated in FIGS. 1 and 2 and incorporated herein by reference.

In a preferred embodiment the shape of the beam spot on the treated area is an apparent circle, which is actually a rotating diameter by a line of light. U.S. Pat. No. 7,922,751, incorporated herein by reference, discloses a device to sweep such a circular beam spot. The device disclosed in that patent can be programmed to move the scanning head in a manner to achieve any desired shape of a treatment zone on the head of a patient. A sample selection of available scan patterns is shown in that patent at FIGS. 8a-h.

### Alzheimer's Example

In one example light therapy is applied to patients with mild to moderate Alzheimer's disease improve their memory, thinking and behaviors. The light-emitting device is a mains powered variable hertz laser device made up of five independent red laser diodes mounted in scanner devices and positioned equidistant from each other. Each scanner emits 7.5 milliwatts (m)±1.0 mW 640 nanometers (nm) with a tolerance of ±10 nm of red laser light.

The laser energy is administered to each patient's frontal cortex, temporal regions and base of the skull 8 times across 4 consecutive weeks, 2 times each week, for 10 minutes of treatment time during each of 8 treatments. The total energy delivered to the patient per procedure administration is 22.5 joules. The patient's frontal cortex, temporal regions and the base of the skull are lazed equally in a continuous sweeping motion continuously during the 10-minute treatment session using pulse frequencies of 8 Hz, 53 Hz, 73 Hz and 101 Hz. The laser energy is applied four inches from the skin surface, and the laser light is directed perpendicular to the plane of the skin ensuring that the beam is penetrating perpendicular to the skin.

### Autism Example

Children exhibiting autistic behaviors are treated with low-level laser by scanning each patient's frontal cortex, temporal area and the base of the skull for 10 minutes, twice weekly, 3-4 days apart, for three consecutive weeks, for a total of 6 treatments. The patient's frontal cortex, temporal regions and the base of the skull are lazed equally in a continuous sweeping motion continuously during the 10-minute treatment session using pulse frequencies of 8 Hz, 53 Hz, 73 Hz and 101 Hz. The light-emitting device used comprised a hand-held laser device with two 7 mW red semiconductor laser diodes emitting a line of red laser light at a wavelength of 635 nm+/−5 nm. Changes in behavior are measured using the Aberrant Behavior Checklist (“ABC”). Significant improvements are achieved in all 5 ABC Subscales and the ABC global score. No adverse events occurred.

While there has been illustrated and described what is at present considered to be the preferred embodiments of the present invention, it will be understood by those skilled in the art that various changes and modifications may be made and equivalents may be substituted for elements thereof without departing from the true scope of the invention. Therefore, it is intended that this invention not be limited to the particular embodiments disclosed, but that the invention will include all embodiments falling within the scope of the appended claims.

1. A method of treating a brain disease in a patient comprising applying light energy to the patient's head above the Reid line and at a pulse frequency of brain waves emanating from a healthy brain.

2. The method of claim 1 further comprising activating one or more cranial nerves while applying the light energy.

3. The method of claim 1 wherein the light energy has a wavelength in the red range.

4. The method of claim 1 wherein the light energy has a wavelength of 640 nm nominal.

5. (canceled)

6. The method of claim 1 wherein the light energy has a pulse frequency of delta brain waves.

7. The method of claim 1 wherein the light energy has a pulse frequency of theta brain waves.

8. The method of claim 1 wherein the light energy has a pulse frequency of alpha brain waves.

9. The method of claim 1 wherein the light energy has a pulse frequency of beta brain waves.

10. The method of claim 1 wherein the light energy has a pulse frequency of gamma brain waves.

11. A method of treating a brain disease in a patient consisting of:
a. determining the type of brain disease; and
b. applying light energy to a patient's head above the Reid line, wherein the light energy is:
i. laser light at 640 nm nominal; and
ii. applied at a pulse frequency of one or more of delta, theta, alpha, mu, beta or gamma brain waves.

12. A method of treating a brain disease in a patient consisting of applying light energy to the scalp of a patient near the frontal cortex and temporal lobe of the patient's brain at a pulse frequency of brain waves emanating from a healthy brain.

13. The method of claim 12 wherein the light energy has a wavelength of 640 nm nominal.

14. A method of treating a brain disease in a patient comprising applying light energy having a wavelength of 640 nm nominal to the scalp of a patient above the Reid line and at a pulse frequency of brain waves emanating from a healthy brain.

15. The method of claim 14 wherein the light energy is applied near the motor cortex of the patient's brain.

16. The method of claim 14 wherein the light energy is applied near the cortex of the patient's brain.

17. The method of claim 14 wherein the light energy is applied near the basal ganglia of the patient's brain.

18. The method of claim 14 wherein the light energy is applied near the frontal lobe of the patient's brain.

19. The method of claim 14 wherein the light energy is applied at a pulse frequency of one or more of delta, theta, alpha, mu, beta or gamma brain waves.

20. The method of claim 14 further comprising activating one or more cranial nerves while applying the light energy.
